(19) United States
(12) Patent Application Publication
(10) Pub. No.: US 2024/0382126 A1
(43) Pub. Date: Nov. 21, 2024

(54) EMOTION ESTIMATION DEVICE AND METHOD OF GENERATING EMOTION ESTIMATION MODEL

(71) Applicant: DENSO TEN Limited, Kobe-shi, Hyogo (JP)

(72) Inventors: Tetsuhiro KATO, Kobe (JP); Kimitaka MURASHITA, Kobe (JP)

(73) Assignee: DENSO TEN Limited, Kobe-shi, Hyogo (JP)

(21) Appl. No.: 18/693,240

(22) PCT Filed: Dec. 27, 2022
(86) PCT No.: PCT/JP2022/048421

§ 371 Date: Mar. 19, 2024

(30) Foreign Application Priority Data
Dec. 28, 2021 (JP) 2021-214570

(51) Int. Cl. A61B 5/16; A61B 5/0205

(52) U.S. Cl. CPC A61B 5/165; A61B 5/0205

(57) ABSTRACT

An emotion estimation device includes controller and emotion estimation model. In the emotion estimation model, four combination states in which two brain wave states obtained by stratifying first index based on ratio of β wave to α wave in brain wave by first determination threshold and two heart beat states obtained by stratifying second index based on heart beat low-frequency component by second determination threshold are combined are formed, and emotion type is set to each of four combination states, and controller determines brain wave state by stratifying first index calculated based on brain wave acquired from subject by first determination threshold, determines heart beat state by stratifying second index calculated based on heart rate acquired from subject by second determination threshold, and determines combination state in model corresponding to determined brain wave state and heart beat state, and determines emotion type corresponding to determined combination state as estimated emotion.

### FIELD

The present invention relates to an emotion estimation device and a method of generating an emotion estimation model.

### BACKGROUND

A technique of estimating an emotion of a subject by mapping information obtained from the heart waveform (electrocardiographic waveform) of the subject to the Russell circumplex model has been known (for example, refer to Patent Literature 1).

### CITATION LIST

### Patent Literature

Patent Literature 1: JP-A-2019-63324

### SUMMARY

### Technical Problem

However, in the conventional technique, there is a problem that it is difficult to estimate an emotion based on physiological signals with high accuracy.

The Russell circumplex model is a model in which emotion types are placed on a circular plane centered at the origin of a coordinate plane having arousal on a vertical axis (AROUSAL) and valence indicating a degree of pleasantness or unpleasantness on a horizontal axis (VALENCE).

In this Russel circumplex model, by plotting arousal and valence of the subject on the Russell circumplex model coordinate plane, an emotion type of the subject is estimated.

The arousal and valence in the Russel circumplex model are psychological constructs, and there are many challenges to realizing it as an actual emotion estimation device. For example, in the Russel circumplex model, it is necessary to estimate the arousal and the valence of a subject, but to realize as an emotion estimation device, some kind of physiological signals of the subject are to be measured, and the arousal and the valence are to be estimated from the measurement values. However, for the point of which physiological signals should be processed by what process to estimate these arousal and valence, a method has not been established, and it is a major challenge to realizing an emotion estimation device.

Therefore, proposals for emotion estimation devices based on the Russell Circumplex Model have conventionally been made, but estimation of emotions of subjects with high accuracy has been difficult.

The present invention has been achieved in view of the above problems, and it is an object thereof to estimate emotions highly accurately.

### Solution to Problem

An emotion estimation device to estimate an emotion, includes: a controller; and an emotion estimation model, wherein in the emotion estimation model, four combination states in which two brain wave states obtained by stratifying a first index based on a ratio of β wave to α wave in a brain wave by a first determination threshold and two heart beat states obtained by stratifying a second index based on a heart beat low-frequency component by a second determination threshold are combined are formed, and an emotion type is set to each of the four combination states, and the controller determines the brain wave state by stratifying the first index calculated based on a brain wave acquired from a subject by the first determination threshold, determines the heart beat state by stratifying the second index calculated based on a heart rate acquired from the subject by the second determination threshold, and determines the combination state corresponding to the determined brain wave state and heart beat state in the emotion estimation model, and determines an emotion type corresponding to the determined combination state as an estimated emotion.

### Advantageous Effects of Invention

According to the present invention, because an emotion is estimated according to a first index and a second index based on physiological signals of a subject, by appropriately setting states of the first index and the second index based on scientific (medical) evidence or experimental results, and by appropriately setting an emotional type corresponding to a combination state of a state of the first index and a state of the second index based on scientific (medical) evidence or experimental results, it is possible to estimate an emotion highly accurately.

### BRIEF DESCRIPTION OF DRAWINGS

FIG. 1 is a diagram illustrating a configuration example of an estimation system according to a first embodiment.

FIG. 2 is a diagram illustrating a configuration example of a server according to the first embodiment.

FIG. 3 is a diagram illustrating an example of a data table that stores emotion type information.

FIG. 4 is a diagram explaining an extraction method of an emotion type.

FIG. 5 is a diagram explaining generation method of a model.

FIG. 6 is a diagram illustrating an example of a coordinate plane of a model.

FIG. 7 is a diagram illustrating an example of a coordinate plane of a model.

FIG. 8 is a diagram illustrating an example of a result display screen of the first embodiment.

FIG. 9 is a flowchart illustrating a flow of extraction processing.

FIG. 10 is a flowchart illustrating a flow of estimation processing.

FIG. 11 is a diagram illustrating an example of an analysis support screen.

FIG. 12 is a diagram explaining a method of identifying an index.

FIG. 13 is a diagram illustrating an example of a result display screen according to the second embodiment.

FIG. 14 is a diagram illustrating an example of a result display screen according to a third embodiment.

### DESCRIPTION OF EMBODIMENTS

Hereinafter, embodiments of an emotion estimation device and method of generating an emotion estimation model disclosed in the present application will be explained in detail with reference to the accompanying drawings. The embodiment described below are not intended to limit the present invention.

### First Embodiment

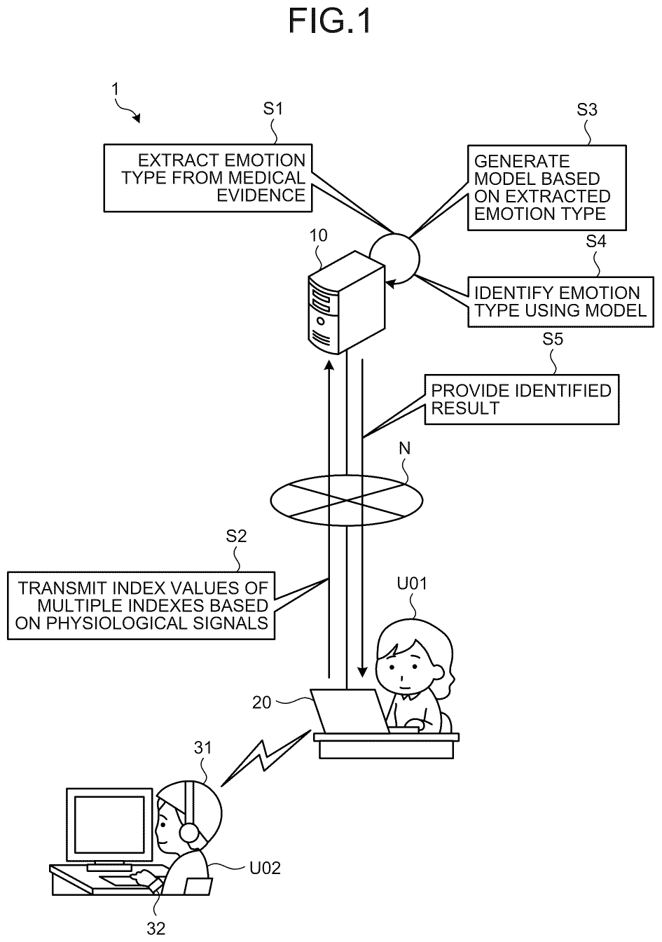

First, an estimation system according to a first embodiment will be explained using FIG. 1. FIG. 1 is a diagram illustrating a configuration example of the estimation system according to the first embodiment.

As illustrated in FIG. 1, an estimation system 1 includes a server 10, a terminal device 20, a sensor 31, and a sensor 32. The estimation system 1 estimates an emotion of a subject U02.

The subject U02 is, for example, an esports player. The estimation system 1 estimates an emotion of the subject U02 that is playing a video game. In the explanation of the present embodiment, to make the explanation more specific and easily understandable, an application scenario for esports described above is assumed as one application example, and it will be explained with state transitions and the like.

An estimation result of emotions is used, for example, for mental training of the subject U02 in esports. For example, as for situations in which the subject U02 experiences negative emotions (anxiety, anger, or the like) during a competition, it is determined that intensive training customized to address the emotional states is necessary.

As for another application example, the subject U02 may be a patient in a medical institution. In this case, an emotion estimated by the estimation system 1 is used for inspections, treatments, and the like.

For example, when the subject U02, which is a patient, is feeling anxious, a staff at a medical institution can take measures, such as counseling.

Furthermore, the subject U02 may be a student at an educational institution. In this case, an emotion estimated by the estimation system 1 is used to improve the content of the class.

For example, when the subject U02, which is a student, is feeing bored in a class, the teacher improves the content of the class to make it more interesting for the student.

Moreover, the subject U01 may be a driver of a vehicle. In this case, an emotion estimated by the estimation system 1 is used to promote safe driving.

For example, when the subject U02, which is a driver, is not feeling an appropriate level of tension while driving, an in-vehicle device outputs a message to prompt the driver to focus on driving.

Moreover, the subject U02 may be a viewer or listener of content, such as video and music. In this case, an emotion estimated by the estimation system 1 is used to generate more contents.

For example, a video content provider can create a highlight video by collecting scenes that the subject U02, which is the viewer, finds enjoyable.

The server 10 and the terminal device 20 are connected through a network N. For example, the network N is the Internet or an intranet.

For example, the terminal device 20 is a personal computer, a smartphone, a tablet computer, or the like. The terminal device 20 is used by an analyst U01.

The sensor 31 and the sensor 32 transmit a detected sensor signal to the terminal device 20.

The sensor 31 is, for example, a headgear-type brainwave sensor. Moreover, the sensor 32 is, for example, a wristband-type heart rate sensor.

For example, the sensor 31 and the sensor 32 establish communication connection with the terminal device 20 in accordance with communication standards such as Wi-Fi (registered trademark) and Bluetooth (registered trademark), and transmit a sensor signal to the terminal device 20.

A flow of processing of the estimation system 1 will be explained using FIG. 1.

The server 10 extracts an emotion type from medical evidences in advance (step S1). The medical evidences are, for example, papers and books. The extraction method of an emotion type will be described later.

The terminal device 20 transmits index values of multiple indexes based on physiological signals to the server 10 (step S2). For example, the terminal device 20 transmits index values of two different indexes relating to brain waves and heart rate.

The index value is an index value relating to a physiological signal. For example, “heart beat interval mean” and “heart beat low frequency (LF) component” are index values. Moreover, specific values corresponding to the respective indexes (for example, a numerical value) are index values. The index values are values calculated from sensor values of respective sensors or values calculated from the sensor value.

The server 10 generates a model based on an extracted emotion type (step S3). At this time, the server 10 generates a model that matches an index value received from the terminal device 20. The generation method of a model will be described later.

The server 10 then identifies an emotion type from the index value by using the generated model (step S4). The server 10 provides the identification result of an emotion type to the terminal device 20 (step S5).

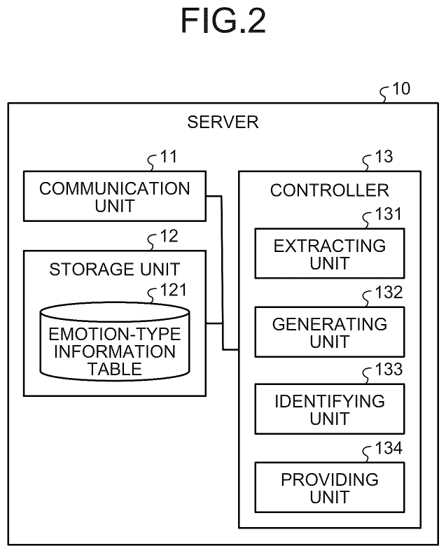

FIG. 2 is a diagram illustrating a configuration example of a server according to the first embodiment. The server 10 is one example of a computer that performs the generation method. Moreover, the server 10 is one example of the emotion estimation device.

As illustrated in FIG. 2, the server 10 includes a communication unit 11, a storage unit 12, a control unit 13, and a so-called controller 13.

The communication unit 11 is an interface to perform data communication with other devices through the network N. The communication unit 11 is, for example, a network interface card (NIC).

The storage unit 12 and the controller 13 of the server 10 are implemented by, for example, a computer having a central processing unit (CPU), a read-only memory (ROM), a random access memory (RAM), a flash memory, an input/output port, and the like, or various kinds of circuits.

The CPU of the computer serves as an extracting unit 131, a generating unit 132, an identifying unit 133, and a providing unit 134 of the controller 13 by reading and executing a program stored, for example, in an ROM.

Moreover, the storage unit 12 corresponds to a RAM or a flash memory. The RAM or the flash memory stores an emotion-type information table 121 and the like.

The server 10 may acquire the programs described above or various kinds of information through other computers (servers) connected via a wired or wireless network or through a portable recording medium.

The emotion-type information table 121 is information in which information relating to an index and an emotion type are associated with each other. Overviews of respective items of the emotion-type information table 121 will be explained herein. A generation method of use method of the emotion-type information table 121 will be described later.

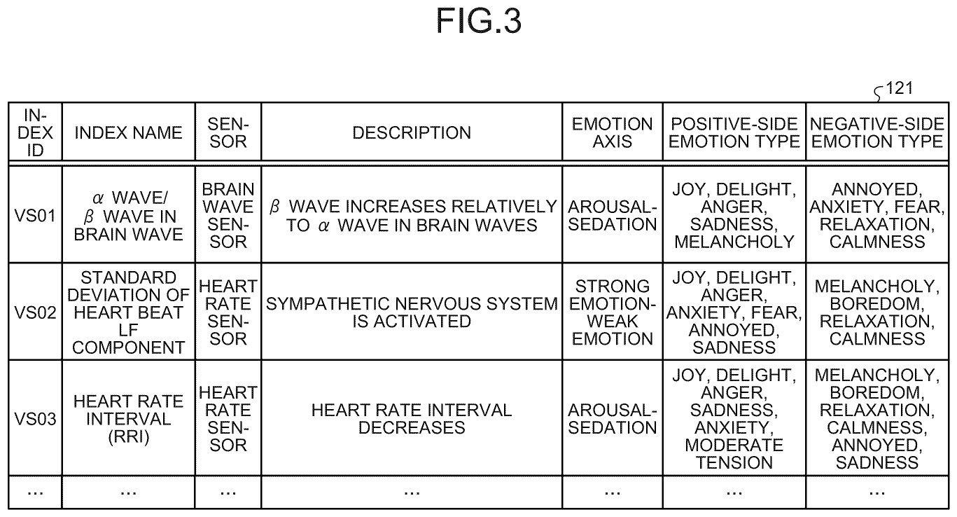

FIG. 3 is a diagram illustrating an example of a data table that stores emotion type information. As illustrated in FIG. 3, items of the emotion-type information table 121 includes an index ID, an index name, a sensor, positive/negative, description, an emotion axis, and positive-side and negative-side emotion types, and these data constitute the emotion type information. To make explanation easily understandable, hereinafter, when data (value) itself in each item is expressed, it will be expressed with a term “data” added thereto. For example, “index ID data” indicates values themselves, such as “VS01” and “VS02”.

The index ID of the emotion-type information table 121 is a character string serving as an ID to identify an index (character strings are stored).

Data of this index ID serves as primary key data, that is, records are structured for each of the index ID data, and in the record, index name data, sensor data, description data, emotion axis data, positive-side emotion type data, and negative-side emotion type data associated with the index ID data are stored.

“INDEX NAME” in the emotion-type information table 121 is information indicating a name of an index (character string).

“SENSOR” in the emotion-type information table 121 is information to identify a sensor necessary for acquiring an index value to be a corresponding index. For example, for the index ID being VS01 (record), the sensor to be used is a brainwave sensor.

In processing described later, an index is used as an axis constituting a coordinate plane. Moreover, in the processing, whether an index value is in a positive direction (larger than) or in a negative direction (smaller than) relative to the origin (determination threshold) holds significance.

“POSITIVE SIDE EMOTION TYPE DATA” and “NEGATIVE-SIDE EMOTION TYPE DATA” in the emotion-type information table 121 are information indicating whether data corresponding to cases in which an index value of the record is a positive value and is a negative value is stored.

Therefore, index values are standardized such that a mean value is to be 0. That is, when a physiological signal shows the mean value, based on an estimation that no emotion associated with the physiological signal has occurred, the above standardization is performed. For index values, not limited to the standardization described above, it is only required to be converted to a form easy to handle on a coordinate plane or a form in which the accuracy of an estimation result becomes high (correction based on an evaluation result of the emotion estimation result, or the like) or the like. When an index value is standardized, the determination threshold to stratify the state of an index is 0.

Moreover, the server 10 may change the standardization method or the transformation method of index values according to a purpose of using an estimated emotion, or the like. For example, because between the contexts of esports and driving, the level of an emotional influence is different (in driving of vehicles, a high level of composure is required for safe driving compared to e-sports), the server may change the standardization method or the transformation method, for example, to determine an excited emotion based on a lower determination threshold.

Furthermore, instead of transforming the form of an index value, the position of the origin on the coordinate plane may be adjusted according to the index value.

“DESCRIPTION” in the emotion-type information table 121 is explanation about the relevant record. For example, it is explanation about a relationship among data of the respective items of the relevant data.

“EMOTION AXIS” in the emotion-type information table 121 is a character string used as a label when an index is used as an axis on the coordinate plane. In the example illustrated in FIG. 3, the index name data indicates a meaning of an emotion axis when it takes a value on respective directional side of positive/negative values.

“POSITIVE-SIDE EMOTION TYPE” and “NEGATIVE-SIDE EMOTION TYPE” in the emotion-type information table 121 is a set of keywords expressing an emotion type corresponding to the relevant record, and indicates an emotion type experienced by the subject when the index name data takes a value on respective directional side of positive/negative data.

For example, in the record on the first row (record of the index ID data, “VS01”) of the emotion-type information table 121 in FIG. 3, “β WAVE/α WAVE IN BRAIN WAVE” is stored as the index name data, “BRAINWAVE SENSOR” is stored as the sensor type data to acquire an index value of the relevant index, “β WAVE INCREASES RELATIVELY TO α WAVE IN BRAIN WAVES” (positive side) is stored as the description data for the positive side, “AROUSAL-SEDATION” is stored as the emotion axis data, “JOY, DELIGHT, ANGER, SADNESS, MELANCHOLY” is stored as the emotion type data on the positive side, and “ANNOYED, ANXIETY, FEAR, RELAXATION, CALMNESS” is stored as the emotion type data on the negative side.

In other words, the index ID data being “VS01” is an index to measure brain waves using a “BRAINWAVE SENSOR”. When the measurement result (index value) shows that “β WAVE INCREASES RELATIVELY TO α WAVE IN BRAIN WAVES” is positive, it is estimated to be in the “AROUSAL” state and to have a possibility that an emotion, such as “JOY, DELIGHT, ANGER, SADNESS, MELANCHOLY”, is being experienced. Moreover, when the measurement result (index value) shows that “β WAVE INCREASES RELATIVELY TO α WAVE IN BRAIN WAVES” is not true (negative), it means that it is estimated to be in the “SEDATION” state, and to have a possibility that an emotion, such as “ANNOYED, ANXIETY, FEAR, RELAXATION, AND CALMNESS”, is being experienced.

Processing of the respective components of the controller 13 will be explained. The main processing by the extracting unit 131, the generating unit 132, the identifying unit 133, and the providing unit 134 can be referred to as controller 13.

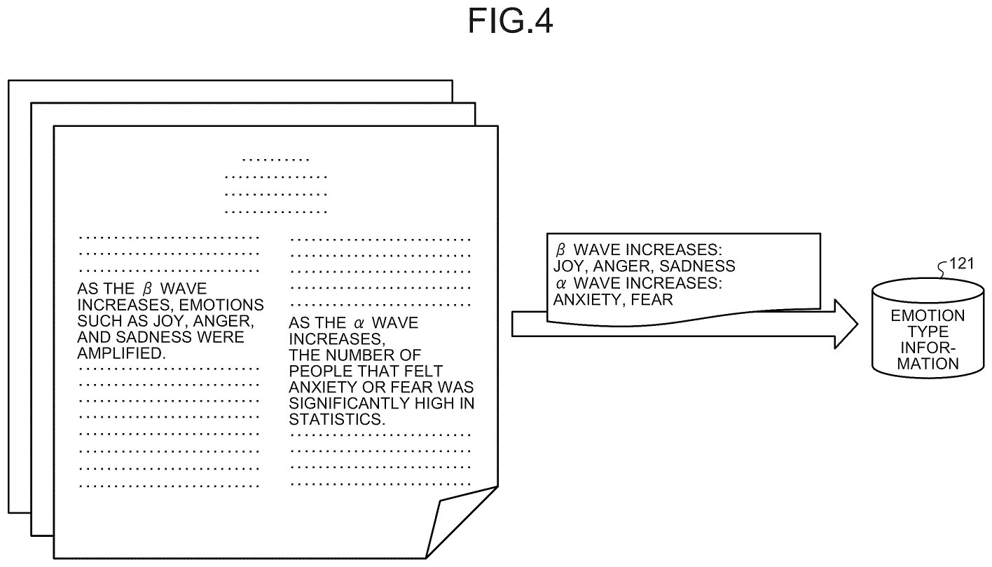

The extracting unit 131 extracts an emotion type associating with an index based on a medical evidence. FIG. 4 is a diagram explaining an extraction method of an emotion type.

As illustrated in FIG. 4, the extracting unit 131 extracts information relating to an index and an emotion type by performing natural language analysis on texts written in papers, books, and the like. For example, a first emotion-type information or a second emotion-type information is generated by language analysis of a document in which a medical evidence is described.

The extracting unit 131 may perform natural language analysis by an existing machine learning method. Moreover, processing of extracting an emotion type associating with an index from medical evidence may be performed manually. In this case, the extracting unit 131 extracts information relating to an index and an emotion type based on input information by an operator.

In the example in FIG. 4, the extracting unit 131 extracts emotion types, “JOY”, “ANGER”, and “SADNESS”, associating with the increase of β wave, based on text, “As the β wave increases, emotions such as joy, anger, and sadness were amplified”.

Moreover, in the example in FIG. 4, the extracting unit 131 extracts emotion types, “ANXIETY” and “FEAR”, associating with the increase of α wave, based on text, “As the α wave increases, the number of people that felt anxiety or fear was significantly high in statistics”.

Furthermore, although an example of an evidence is omitted, similarly to the above, for example, “AROUSAL” is extracted associating with the emotion axis, for example, based on text, “In the arousal state, the β wave increases compared to the α wave in brain waves”.

Note that which of the β wave and the α wave increases is one example of an index based on brain waves, which is one of the physiological signals. Moreover, based on such information extracted by the extracting unit 131, the emotion-type information table 121 is generated. In the example described above, for the record of the index ID data, “VS01” (the index ID data is set appropriately such that identical value do not exist), “β WAVE/α WAVE OF BRAIN WAVE” is stored as the index name data, “BRAINWAVE SENSOR” is stored as the sensor data, “β WAVE INCREASES RELATIVELY TO α WAVE IN BRAIN WAVES” is stored as the description data, “AROUSAL-SEDATION” is stored as the emotion axis data, “JOY, DELIGHT, ANGER, SADNESS, MELANCHOLY” is stored as the emotion type data on the positive side, and “ANNOYED, ANXIETY, FEAR, RELAXATION, CALMNESS” is stored as the emotion type data on the negative side. That is, these extracted emotion types are to be emotion type candidates for each index, such as the first index based on a ratio between the β wave and the α wave of brain waves set in the emotion estimation model.

The generating unit 132 acquires the first emotion-type information associated with the first index based on a physiological signal from the information extracted by the extracting unit 131, and acquires the second emotion-type information associated with the second index based on an physiological signal. The generating unit 132 generates the emotion estimation model (empty emotion estimation model in which no emotion types are set) in which an emotion type selected from the first emotion-type information and the second emotion-type information can be associated according to respective combination state constituted of a combination of a first index state, which is each state in the first index, and a second index state, which is each state in the second index state, that is, according to the combined first index state and second index state.

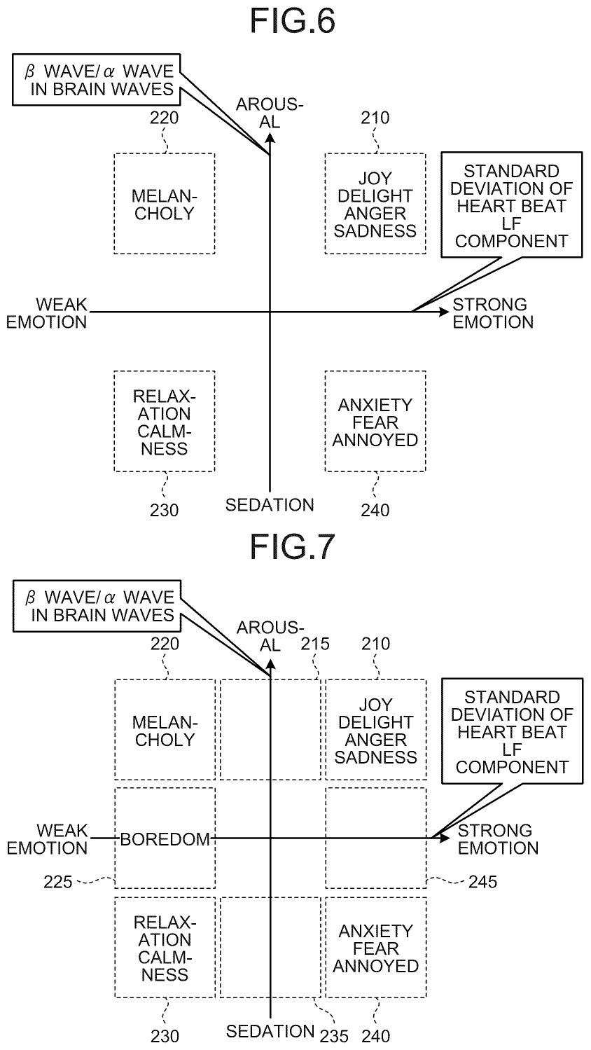

When the emotion estimation model thus generated is visualized, it becomes the emotion estimation model represented by the emotion map illustrated in FIG. 6. In FIG. 6, the first index is the vertical axis, and the second index is the horizontal axis. The first index and the second index are respectively stratified by a threshold (0 when it is standardized), and respectively turn into two stratified states. The combination states that are combinations of the respective stratified states of the first index and the second index correspond to a first to a fourth quadrants of an emotion map, and this corresponds to the empty emotion estimation model. By arranging (setting) emotion candidates corresponding to these respective quadrants, the emotion estimation model is formed. Hereinafter, it will be explained based on a visualized emotion map.

The generating unit 132 acquires emotion types that correspond to specified two or more indexes, that is, duplicated emotion types (for example, the first emotion type and the second emotion type), from the emotion-type information table 121 in which indexes relating to physiological signals (for example, the first index and the second index) and emotion types are associated.

Subsequently, the generating unit 132 generates a model (emotion estimation model) in which the acquired emotion types described above are arranged in each quadrant of space defined by the axes associated with the respective two indexes.

The space herein refers to Euclidean space. That is, the space herein includes two dimensional plane and three or more dimensional space.

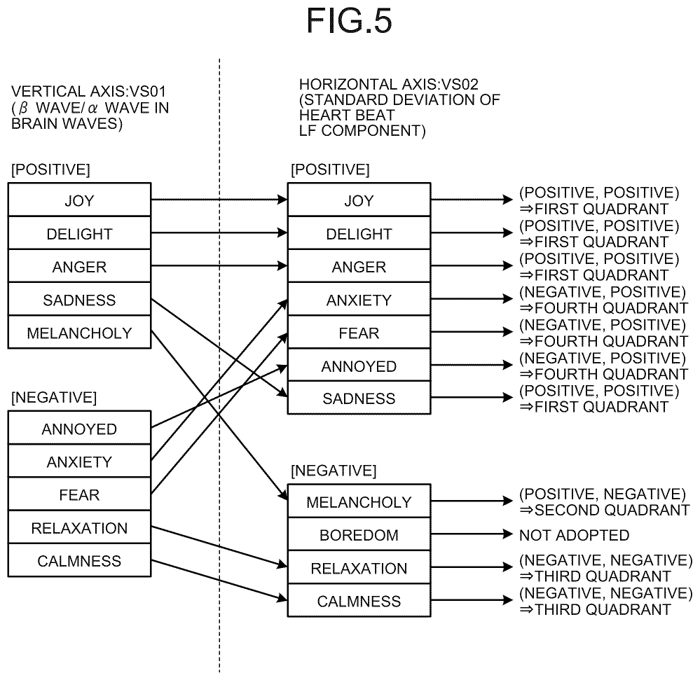

The generation method of a model by the generating unit 132 will be specifically explained using FIG. 5. FIG. 5 is a diagram explaining the generation method of a model.

It is assumed that β wave/α wave in brain wave (index “VS01”) and is specified as an index, and a standard deviation of a heart beat LF component (index “VS02”) are specified.

The generating unit 132 refers to the emotion-type information table 121, and acquires various kinds of data respectively from the record of the index ID “VS01” and the record of the index ID “VS02”.

As illustrated in FIG. 5, the generating unit 132 assigns the index “VS01” to the vertical axis, and the index “VS02” to the horizontal axis. The assignment of axes may be reversed to the one illustrated in FIG. 5. Specifically, because the emotion axis data of the index “VS01” is “AROUSAL-SEDATION”, the vertical axis is “arousal-sedation (positive side-negative side)”, and because the emotion axis data of the index “VS02” is “STRONG EMOTION-WEAK EMOTION”, the horizontal axis is “strong emotion-weak emotion (positive side-negative side)”.

Next, the generating unit 132 analyzes relationships between positive-side emotion type data and a negative-side emotion type data of the index “VS01” of respective emotion types and between positive-side emotion type data and a negative-side emotion type data of the index “VS02” of an identical emotion type.

The generating unit 132 arranges the respective emotion types in the respective quadrants on the two-dimensional coordinate plane defined by the vertical axis and the horizontal axis based on the analyzed relationships.

Specifically, the generating unit 132 arranges emotion type data that are present in common to the positive-side emotion type data of the index “VS01” and the positive-side emotion type data of the index “VS02” in the first quadrant.

Moreover, the generating unit 132 arranges emotion type data present in common to the negative-side emotion type data of the index “VS01” and the positive-side emotion type data of the index “VS02” in the fourth quadrant.

Furthermore, the generating unit 132 arranges emotion type data present in common to the negative-side emotion type data of the index “VS01” and the negative-side emotion type data of the index “VS02” in the third quadrant.

Moreover, the generating unit 132 arranges emotion type data that are present in common to the positive-side emotion type data of the index “VS01” and the negative-side emotion type data of the index “VS02” in the second quadrant.

For example, as illustrated in FIG. 3, the emotion type “JOY” of the index “VS01” is included in the positive-side emotion type data, and the emotion axis data of the index “VS01” is “AROUSAL-SEDATION (positive side-negative side)”. Moreover, the emotion type “JOY” of the index “VS02” is included in the positive-side emotion type data, and the emotion axis data of the index “VS02” is “STRONG EMOTION-WEAK EMOTION (positive side-negative side).

Therefore, the generating unit 132 arranges the emotion type “JOY” in the first quadrant in the two-dimensional Euclidean space formed with the vertical axis “AROUSAL-SEDATION (positive side-negative side)” and the horizontal axis “STRONG EMOTION-WEAK EMOTION (positive side-negative side)” as illustrated in FIG. 6.

As illustrated in FIG. 5, the generating unit 132 determines not to adopt emotion type data that is not included as emotion type data in at least one index out of selected two kinds of indexes, that is, for example, emotion type “BOREDOM” not included in the index “VS01” (but included in “VS02”), and does not arrange it on the coordinate plane at this point. In other words, only emotion type data that is included in both of the selected two kinds of indexes is arranged on the coordinate plane. A method in which such an emotion type to be handled as not adopted is arranged on the coordinate plane is also conceivable, but the method will be described later.

As described, the generating unit 132 forms a two-dimensional coordinate plane defined by the first emotion axis associated with index data (first emotion-index data) representing a first emotion and the second emotion axis associated with index data (second emotion-index data) representing a second emotion. The generating unit 132 determines a position relative to the first emotion axis of the respective emotion type data associated with the first emotion-index data (in the example of FIG. 3, the positive-side emotion type is on the positive side/the negative-side emotion type is on the positive side). Furthermore, the generating unit 132 determines a position relative to the second emotion axis of the second emotion-type data associated with the second emotion-index data. The generating unit 132 then generates a model in which respective emotion-type data is arranged in each quadrant of the formed two-dimensional coordinate plane based on the positions relative to the respective emotion axes of the first emotion-type data and the second emotion-type data.

Thus, a model that enables to identify an emotion type based on different emotion axes including the first emotion axis (for example, arousal) and the second emotion axis (for example, intensity of emotion) can be generated.

The first index or the second index may be an index indicating an arousal state other than the above (VS01). The first index or the second index may be an index indicating an intensity of emotion other than the above (VS02).

### Model Example 1

It has been known that the arousal state affects the autonomic nervous system (sympathetic nervous system and parasympathetic nervous system), to change the contractility of the heart, and heart beat intervals are affected thereby. As the emotion types that lead to the arousal state, “JOY”, “DELIGHT”, “ANGER”, “ANXIETY”, and “MODERATE TENSION” have been known. Moreover, as the emotion types that lead to the sedation state, “MELANCHOLY”, “BOREDOM”, “RELAXATION”, “CALMNESS”, “ANNOYED”, and “SADNESS” have been known. Based on medical evidence indicating these facts, the emotion-type information table 121 is generated.

An example of this is a record of the index ID data “VS03” in the emotion-type information table 121, and “HEART BEAT INTERVAL (RRI)” is stored as the index name data, “HEART RATE SENSOR” is stored as the sensor data, “HEART BEAT INTERFACE DECREASES” is stored as description data, “AROUSAL-SEDATION” is stored as the emotion axis data, “JOY, DELIGHT, ANGER, ANXIETY, MODERATE TENSION” is stored as the positive-side emotion type data, and “MELANCHOLY, BOREDOM, RELAXATION, CALMNESS, ANNOYED, SADNESS” is stored as the negative-side emotion type data.

On the other hand, it has been known that a standard deviation of heart beat LF component indicates a level of activity of the sympathetic nervous system and the parasympathetic nervous system.

The activity of the sympathetic nervous system is known to have a correlation with intense emotions, while the activity of the parasympathetic nervous system has a correlation with weak emotions. As the emotion types to be a strong emotion, “JOY”, “DELIGHT”, “ANGER”, “ANXIETY”, “FEAR”, “ANNOYED”, and “EXCITEMENT” have been known. Moreover, as the emotion types to be a weak emotion, “MELANCHOLY”, “BOREDOM”, “RELAXATION”, and “CALMNESS” have been known. Based on medical evidence indicating these facts, the emotion-type information table 121 is generated.

An example of this is the record of the index ID data “VS02” in the emotion-type information table 121, and “STANDARD DEVIATION OF HEART BEAT LF COMPONENT” is stored as the index name data, “HEART RATE SENSOR” is stored as the sensor data, “SYMPATHETIC NERVOUS SYSTEM IS ACTIVATED” is stored as the description data, “STRONG EMOTION-WEAK EMOTION” is stored as the emotion axis data, “JOY, DELIGHT, ANGER, ANXIETY, FEAR, ANNOYED, SADNESS” is stored as the positive-side emotion type data, and “MELANCHOLY, BOREDOM, RELAXATION, CALMNESS” is stored as the negative-side emotion type data.

The generating unit 132 assigns “AROUSAL-SEDATION” to the vertical axis, and “STRONG EMOTION-WEAK EMOTION” to the horizontal axis.

The generating unit 132 arranges each emotion type in a corresponding quadrant in two-dimensional Euclidean space formed with the vertical axis “AROUSAL-SEDATION (positive side-negative side) and the horizontal axis “STRONG EMOTION-WEAK EMOTION (positive side-negative side)” based on analysis processing to determine to which of the positive-side emotion type and the negative-side emotion type, each emotion type of the index ID data “VS03” and “VS02” is assigned.

The generating unit 132 arranges the emotion types “JOY”, “DELIGHT”, “ANGER”, and “ANXIETY” in the first quadrant, “ANXIETY” and “ANNOYED” in the third quadrant, and “MELANCHOLY”, “BOREDOM”, “RELAXATION”, and “CALMNESS” in the fourth quadrant.

### Model Example 2

Because heart beat intervals are significantly influenced by respiration, there is a case in which the accuracy of the model decreases. Therefore, the generating unit 132 may generate a model in which an influence of respiration is avoided by assigning an index relating to a brain wave in the vertical axis.

As the emotion types that lead to the arousal state affected by brain waves, “JOY”, “DELIGHT”, “ANGER”, “SADNESS”, and “MELANCHOLY” have been known. Moreover, as the emotion types that lead to the sedation state affected by brain waves, “MELANCHOLY”, “BOREDOM”, “RELAXATION”, and “CALMNESS” have been known. Based on medical evidence indicating these facts, the emotion-type information table 121 is generated.

An example of this is the record of the index ID data “VS01” in the emotion-type information table 121, and “β WAVE/α WAVE OF BRAIN WAVE” is stored as the index name data, “BRAINWAVE SENSOR” is stored as the sensor data, “β WAVE of BRAIN WAVE INCREASES RELATIVELY” is stored as the description data, “AROUSAL-SEDATION” is stored as the emotion axis data, “JOY, DELIGHT, ANGER, SADNESS, MELANCHOLY” is stored as the positive-side emotion type data, and “ANNOYED, ANXIETY, FEAR, RELAXATION, CALMNESS” is stored as the negative-side emotion type data.

The generating unit 132 assigns “AROUSAL-SEDATION” to the vertical axis, and “STRONG EMOTION-WEAK EMOTION” to the horizontal axis.

The generating unit 132 arranges each emotion type in a corresponding quadrant in two-dimensional Euclidean space formed with the vertical axis “AROUSAL-SEDATION (positive side-negative side) and the horizontal axis “STRONG EMOTION-WEAK EMOTION (positive side-negative side)” based on analysis processing to determine to which of the positive-side emotion type and the negative-side emotion type, each emotion type of the index ID data “VS01” and “VS02” is assigned.

Specifically, the generating unit 132 arranges the emotion types “JOY”, “DELIGHT”, “ANGER”, and “SADNESS” in the first quadrant, “MELANCHOLY” in the second quadrant, “RELAXATION”, and “CALMNESS” in the third quadrant, and “ANXIETY”, “FEAR”, and “ANNOYED” in the fourth quadrant.

As described, the generating unit 132 acquires emotion types respectively corresponding to an index representing a level of arousal based on brain waves, and an index representing an intensity of an emotion based on heart beats.

By using a brainwave sensor capable of estimating the level of arousal based on the β wave/α wave in brain waves from a state of activeness of the cerebral cortex, the influence of respiration is avoided.

The generation method of a model has been explained above, but it will be explained with specific data from a specific example illustrated in FIG. 5. FIG. 6 is a diagram illustrating an example of a coordinate plane of a specific model generated by this specific example.

As illustrated in FIG. 5, the vertical axis of the coordinate plane is the emotion axis “AROUSAL-SEDATION” of the emotion axis of the index ID data “VS01”, and emotion types positioned on the positive side of the vertical axis is the positive-side emotion types, “JOY”, “DELIGHT”, “ANGER”, “SADNESS”, and “MELANCHOLY”, and emotion types positioned on the negative side of the vertical axis is the positive-side emotion types, “ANNOYED”, “ANXIETY”, “FEAR”, “RELAXATION”, and “CALMNESS”.

On the other hand, the horizontal axis of the coordinate plane is the emotion axis “STRONG EMOTION-WEAK EMOTION” of the index ID data, and emotion types positioned on the positive side of the vertical axis are “JOY”, “DELIGHT”, “ANGER”, “ANXIETY”, “FEAR”, and “ANNOYED”, and emotion types positioned on the negative side of the vertical axis are the negative-side emotion types “MELANCHOLY”, “BOREDOM”, “RELAXATION”, and “CALMNESS”.

These are determined based on the emotion type information illustrated in FIG. 3.

The generating unit 132 determines on which side out of the positive side or the negative side, an identical emotion type is positioned on the vertical axis and the horizontal axis, and determines in which quadrant on the coordinate plane of the model it is arranged based on the result. For example, because the emotion type “JOY” is positioned on the positive side of the vertical axis (“AROUSAL-SEDATION”) and on the positive side on the horizontal axis (“STRONG EMOTION-WEAK EMOTION”), the generating unit 132 arranges it in the “first quadrant”.

Bu performing such processing for each of emotion types, as illustrated in FIG. 6, in a region 210 in the first quadrant, the emotion types “JOY”, “DELIGHT”, “FEAR”, and “SADNESS” are arranged, in a region 220 in the second quadrant, the emotion type “MELANCHOLY” is arranged, in a region 230 in the third quadrant, the emotion types “RELAXATION” and “CALMNESS” are arranged, and in a region 240 in the fourth quadrant, the emotion types “ANXIETY”, “FEAR”, and “ANNOYED” are arranged.

Furthermore, as illustrated in FIG. 7, the generating unit 132 may arranged an emotion type that has been handled as not adopted by the method in FIG. 5 in a region between two quadrants. FIG. 7 is a diagram illustrating an example of a coordinate plane of a model.

An emotion type that is not adopted in arrangement on the coordinate plane of the model occurs when relevant emotion type data is not present in at least one of two emotion indexes selected as the two axes on the coordinate plane of the model. In this case, it is determined to be not adopted in the method illustrated in FIG. 5, but as an alternative perspective, when there is no correlation with a specific emotion index (both positive and negative emotion types are not present), it is possible to interpret it as representing an emotion type located at position 0. Accordingly, on the coordinate plane of this model, the generating unit 132 sets a region near the value 0 of the vertical axis and the horizontal axis in addition to the regions of the four quadrants, and arranges an emotion type that has been determined as not to be adopted in the method illustrated in FIG. 5 in either range near the value 0 of the vertical axis and the horizontal axis (based on a positive side and a negative side position of an emotion type in the other index).

In the example described previously, as illustrated in FIG. 7, the generating unit 132 arranges the emotion type “BOREDOM” that has been determined as not adopted in a region 225 between the second quadrant and the third quadrant (handling the emotion type “BOREDOM” as 0 (neutral) with respect to “AROUSAL-SEDATION” axis).

By such a method, the generating unit 132 can arrange even an emotion type that is not adopted in the method illustrated in FIG. 5 in a region 215 between the first quadrant and the second quadrant, in the region 225 between the second quadrant and the third quadrant, in a region 235 between the third quadrant and the fourth quadrant, or in a region 245 between the fourth quadrant and the first quadrant appropriately.

In the above explanation, a method of arranging emotion types in the respective quadrants by language analysis of medical evidence or the like has been presented, but it is also possible to arrange the emotion types in the respective quadrants manually by a developer or the like. For example, physiological signals of a subject are measured under various conditions, and a survey to identify experienced emotion types is conducted. Subsequently, a quadrant in an emotion map is calculated based on the physiological signals measured under various conditions, and the emotion type acquired as a survey result is arranged in the calculated quadrant. By such a method, emotion types can be set in the respective quadrants in the emotion map.

The identifying unit 133 identifies an emotion type of the subject using a model based on a value of an index acquired from a physiological signal of the subject U02. That is, analysis processing is performed on the physiological signal from a sensor worn by the subject U02, to process it into a corresponding index value. By subjecting the physiological signal, two kinds of index values are acquired. The index values are applied to the generated model (on the coordinate plane of the model), and an emotion type in a region to which the index values correspond is identified as an emotion type of the subject U02.

As described, the identifying unit 133 acquires multiple kinds of signals, first physiological signal and second physiological signal, converts the first physiological signal into a first index value, converts the second physiological signal into a second index value, and applies the first index value and the second index value that have been converted from the first physiological signal and the second physiological signal to an emotion model in which an emotion estimation value is determined based on a combination of the first index value and the second index value, to determine the emotion estimation value as an estimated emotion.

The providing unit 134 provides the identified emotion type to the terminal device 20. By providing the identified emotion type by display or the like to the user, such as the subject U02, the user, such as the subject U02, can grasp and estimate the emotion of the subject U02, and can use it for training or the like of the subject U02.

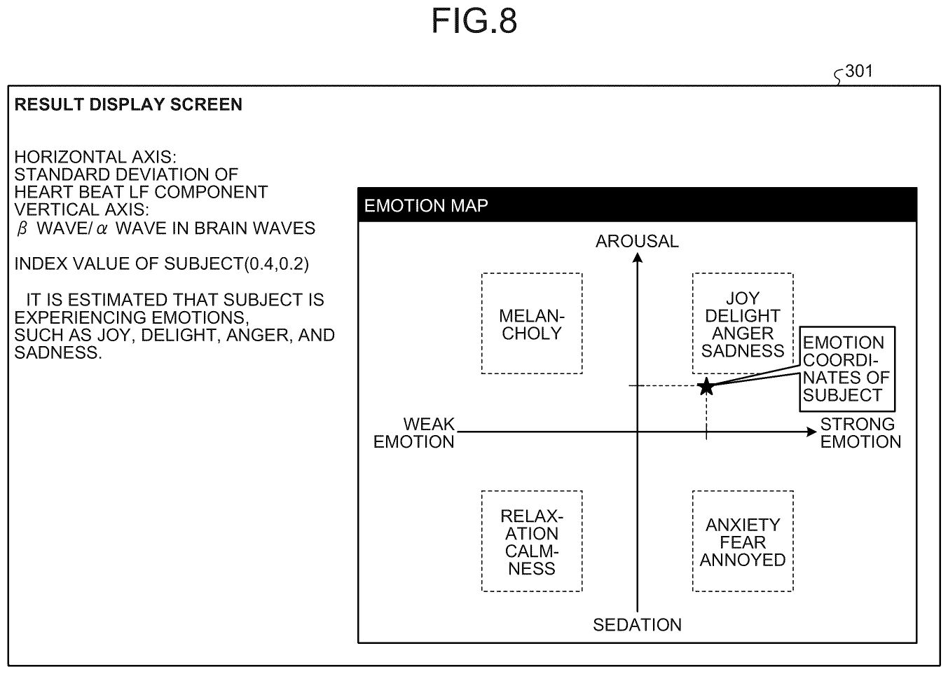

For example, the providing unit 134 displays a result display screen on a display (constituted of a liquid crystal display or the like) of the terminal device 20. FIG. 8 is a diagram illustrating an example of the result display screen.

As illustrated in FIG. 8, on a result display screen 301, indexes assigned to respective axes, index values of a subject, an emotion type relating to an estimated emotion result, text information such as a message, a related image indicating those contents, and the like are displayed. Moreover, on the result display screen 301, an emotion map is displayed. These images are generated by the controller 13 based on calculated index values and an estimated emotion result.

The emotion map shows coordinates (emotion coordinates of the subject) on which index values acquired from physiological signals of the subject U02 are plotted on a coordinate plane of the model.

Because the emotion coordinates are positioned in the first quadrant in the example in FIG. 8, the emotion of the subject U02 is estimated as either one of “JOY”, “DELIGHT”, “ANGER”, and “SADNESS”. Furthermore, based on the plotted position on the emotion map, it becomes possible to estimate the intensity of an emotion type to some extent (an emotion of the emotion type is estimated to be stronger as it is positioned farther from the origin). Moreover, based on the plotted position on the emotion map, it becomes possible to estimate the accuracy of emotion determination (the determination accuracy for the emotion type is estimated to be higher as it is positioned farther from the origin).

An example when two indexes are specified has been explained herein. On the other hand, specified indexes may be three or more. For example, when three indexes are specified, the generating unit 132 arranges an emotion type in either one of eight quadrants (three-dimensional space).

For example, coordinate space is defined, setting emotion axes of the index ID data, “VS01”, “VS02”, and “VS03” as a vertical axis, a horizontal axis, and a depth axis, which are so-called XYZ three-dimensional axes. The identifying unit 133 plots in the coordinate space using three index values based on respective physiological signals, and identifies an emotion type arranged in space of a region in which the plotted point is positioned as an emotion of the subject.

Moreover, although one determination threshold each is used for two indexes is in the example described above, two or more determination thresholds may be used. For example, the first index based on a ratio of β wave to α wave in a brain wave is stratified by two determination thresholds, and the first index may be stratified into three states (brain wave states) based on the first index.

According to the method as described above, the number of regions in which emotion types are arranged increases, and the types of index to be used also increase and, therefore, mode detailed emotion determination is enabled.

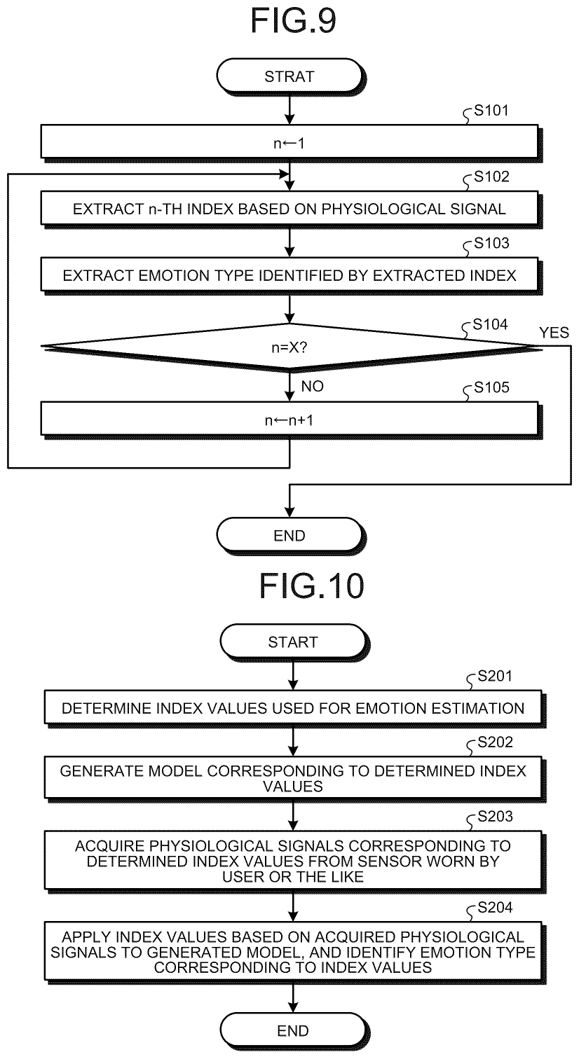

A flow of processing performed by the server 10 will be explained using FIG. 9 and FIG. 10. FIG. 9 is a flowchart illustrating extraction processing performed by the controller 13 (the extracting unit 131). This extraction processing is performed, for example, based on a start instruction by a user, but it is necessary for the user to perform this processing before estimation of an emotion.

Furthermore, FIG. 10 is a flowchart illustrating a flow of the estimation processing. This estimation processing is performed based on a start instruction by the user when the user desires an emotion estimation result.

As illustrated in FIG. 9, at step S101, the controller 13 (the extracting unit 131) substitutes 1 for n, and shifts to step S102. n is a number to identify an index to be extracted. Moreover, X is the number of index. X is set according to the necessity of the user, but the smallest number is 2.

At step S102, the controller 13 (the extracting unit 131) extracts the n-th index based on a physiological signal, and shifts to step S103. At step S103, the controller 13 (the extracting unit 131) extracts data of an emotion type or the like identified by the extracted index, stores it associating with an index ID as the emotion type information as illustrated in FIG. 3, and shifts to step S104.

The controller 13 (the extracting unit 131) extracts index data and emotion type data by performing the natural language analysis with respect to papers and books related to medicine.

Furthermore, the controller 13 (the extracting unit 131) may store relevant data input by a human through an input operation device, such as a keyboard, as the emotion type information, or may acquire relevant data from a socially shared database, such as a medical database, to store it as the emotion type information.

At step S104, the controller 13 (the extracting unit 131) determines whether the number n of index for which the emotion type information is extracted reaches the set number X, and ends the processing if it has reached, and shifts to step S105 if not reached. At step S105, the controller 13 (the extracting unit 131) adds 1 to the counter value n indicating the number of indexes for which the emotion type information is extracted, and returns to step S102. That is, until the number n of index for which the emotion type information is extracted reaches the set number X, processing at step S120 and step S103 is to be repeated.

Moreover, as illustrated in FIG. 10, the controller 13 (the generating unit 132) determines multiple index values to be used for emotion estimation at step S201, and shifts to step S202. This determination is made, for example, by the controller 13 (the providing unit 134) providing, to the user, index information necessary for estimating an emotion that is desired to be estimated by the user, and by the controller 13 (the generating unit 132) taking in an index selected by the user by selection operation.

The controller 13 (the generating unit 132) generates a model corresponding to the determined index value at step S202, and shifts to step S203. The controller 13 (the generating unit 132) can generate a model by the method illustrated in FIG. 5.

The controller 13 (the generating unit 132) acquires an physiological signal corresponding to the determined index value from a sensor worn by the user or the like at step S203, and shifts to step S204. The controller 13 (the generating unit 132) processes the acquired physiological signal as necessary, to convert into an index value.

Moreover, prior to that, the controller 13 (the providing unit 134) provides information about a sensor required to be worn by the user, a guidance for starting the emotion estimation, and the like to the user, to support the user for preparation.

The controller 13 (the generating unit 132) applies the index value based on the acquired physiological signal to the generated model at step S204, identifies an emotion type corresponding to the index value based on the estimation method of an emotion explained in FIG. 6 or the like, and ends the processing. For example, the controller 13 (the generating unit 132) identifies an emotion type based on a quadrant in which the emotion coordinates obtained by plotting the index value are positioned on the coordinate plane of the model.

When the indexes of physiological signals to be used are the index based on the ratio of β wave to α wave in a brain wave (the first index: arousal) and the index based on the heart beat low-frequency component (the second index: intensity of emotion), these actions (actions of the emotion estimation device) are expressed by a configuration of the emotion estimation model (emotion map) included in the emotion estimation device and processing performed by the controller, as follows.

In the emotion estimation model described above, four combination states (the first quadrant to the fourth quadrant) in which two brain wave states (AROUSAL-SEDATION) obtained by stratifying the first index based on the ratio of β wave to α wave in a brain wave by the first determination threshold and two heart beat states (strong emotion-weak emotion) obtained by stratifying the second index based on the heart beat low-frequency component by the second determination threshold are combined are formed, and to the respective four combination states (the first quadrant to the fourth quadrant), emotion types (the first quadrant: “JOY”, “DELIGHT”, “ANGER”, “SADNESS”, the second quadrant: “MELANCHOLY”, the third quadrant: “RELAXATION”, “CALMNESS”, the fourth quadrant: “ANXIETY”, “FEAR”, “ANNOYED”) are set.

The controller determines the brain wave state (AROUSAL-SEDATION) by stratifying the first index calculated based on a brain wave acquired from the subject by the first determination threshold, determines the heart beat state (STRONG EMOTION-WEAK EMOTION) by stratifying the second index calculated based on a heart beat acquired from the subject by the second determination threshold, and determines the combination state (for example, the first quadrant) in the model corresponding to the determined brain wave state and heart beat state, to set an emotion type (for example, “JOY”) corresponding to the determined combination state (for example, the first quadrant) as the estimated emotion (for example, “JOY”).

The server 10 may accept a specification of an emotion type instead of an index, and may generate a model from the specified emotion type. That is, for example, it is a method used when a user wishes to know a state of a certain emotion type (whether it has occurred and its intensity).

In this case, the controller 13 (the generating unit 132) acquires two or more indexes corresponding to a specified emotion type (an emotion type specified as the positive-side emotion type or the negative-side emotion type is included) from the emotion-type information table 121. Moreover, the controller 13 (the generating unit 132) generates a model in which emotion types are arranged in respective quadrants of space defined by axes to which two or more indexes are associated, respectively. The subject wears a necessary sensor based on the emotion-type information table 121, and the controller 13 acquires physiological data from the sensor. Thereafter, based on the physiological data, an emotion of the subject is estimated by a method similar to the method described above.

Thus, even when the analyst U01 lacks sufficient knowledge about the indexes, information estimating a state of the desired emotion type can be acquired.

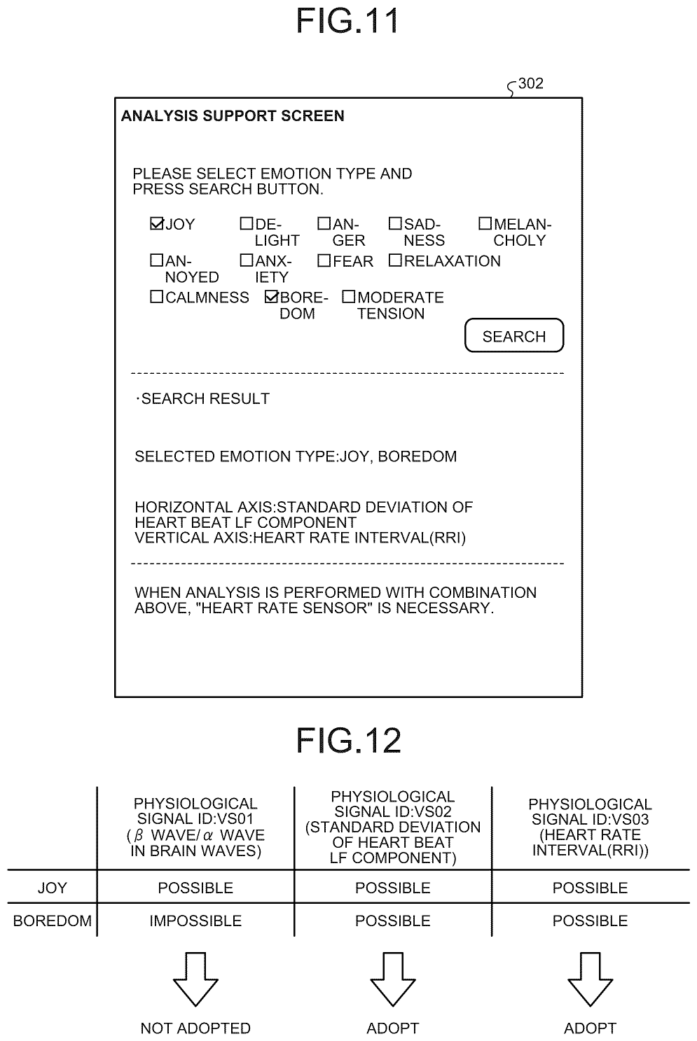

An example of a user interface of such an emotion estimation device will be explained. FIG. 11 is a diagram illustrating an example of an analysis support screen. As illustrated in FIG. 11, on an analysis support screen 302, together with a message, “PLEASE SELECT EMOTION TYPE AND PRESS SEARCH BUTTON”, a check box set with which multiple emotion types are selectable is displayed.

The emotion types displayed with check boxes are data of emotion types included in the positive-side emotion type and the negative-side emotion type in the emotion-type information table 121.

When the search button is pressed, the providing unit 134 identifies a combination of indexes for which estimation of an emotion type selected by the check box is possible, and displays information relating to the identified combination as a search result.

Furthermore, the providing unit 134 provides information relating to a sensor necessary for acquiring the index displayed as the search result.

For example, as illustrated in FIG. 11, on the analysis support screen 302, suppose that the analyst U01 selects the emotion types of “JOY” and “BOREDOM”. Subsequently, based on the respective data in the emotion-type information table 121 illustrated in FIG. 3, “STANDARD DEVIATION OF HEART BEAT LF COMPONENT” and “HEART RATE INTERVAL (RRI) OF VS03” that are indexes including the emotion types of “JOY” and “BOREDOM” (search for the positive-side emotion type and the negative-side emotion type in the emotion-type information table 121) are searched.

On the analysis support screen 302, a combination of “STANDARD DEVIATION OF HEART BEAT LF COMPONENT” and “HEART RATE INTERVAL (RRI)” is displayed as a search result. Furthermore, on the analysis support screen 302, “HEART RATE SENSOR” that is a sensor necessary when analysis is performed with a combination of a standard deviation of the heart beat LF component and the heart rate interval (RRI) is displayed. The user views this screen and prepares the “HEART RATE SENSOR”, to perform emotion estimation.

FIG. 12 is a diagram explaining a method of identifying an index. The example in FIG. 12 is one in the case in which the analyst U01 specifies analysis of emotions of “JOY” and “BOREDOM”. As illustrated in FIG. 12, the providing unit 134 refers to the emotion-type information table 121, and checks whether “JOY” and the “BOREDOM” selected by the analyst U01 are included (POSSIBLE OR IMPOSSIBLE).

The providing unit 134 identifies (adopts) a combination of indexes for which “JOY” and “BOREDOM” are both POSSIBLE, and provides information relating to these identified indexes and information for calculating the indexes to the analyst U01 or the subject.

When more indexes meet the adoption conditions than the number of indexes to be adopted, the providing unit 134 identifies a combination of indexes automatically based on predetermined criteria. For example, criteria, such as the providing unit 134 identifying a combination of indexes that have been frequently selected in the past, identifying a combination of indexes that are estimated to have high accuracy, identifying a combination of indexes for which the required sensor has a high adoption rate, identifying a combination of indexes that enable to identify as many emotion types as possible, can be set.

Moreover, it may be configured such that the providing unit 134 recommends combinations of indexes that can be combined to the analyst U01, and the analyst U01 adopts a combination of indexes selected therefrom.

The physiological signals that can be used in the first embodiment are not limited to those provided in the explanation above. For example, in the emotion-type information table 121, emotion types corresponding to indexes acquired from physiological signals, such as body surface temperature, facial images, and pupil dilation, may be included. Arousal may be a sleepiness level or the like measured by, for example, body temperature, body surface temperature, pupil size, brain wave fluctuations, and image recognition.

Moreover, by providing a configuration that implements functions (function to implement the controller 13) equivalent to the extracting unit 131, the generating unit 132, the identifying unit 133, and the providing unit 134 of the server 10 in the terminal device 20, the terminal device 20 may be configured to perform the emotion estimation actions performed by the server 10 described above. In that case, the terminal device 20 has a configuration equivalent to the server 10 described above.

As described above, the controller 13 of the server 10 according to the first embodiment acquires the first emotion-type information associated with the first index relating to a physiological signal, acquires the second emotion-type information associated with the second index relating to a physiological signal, and generates an emotion estimation model in which an emotion type selected from the first emotion-type information and the second emotion-type information according to the combined first index state and the second index state is associated with each combination index state constituted of a combination of the first index state, which is each state in the first index, and the second index state, which is each state in the second index.

As described, by generating a model according to information in which an index relating to a physiological signal and an emotion type are associated (the emotion-type information table 121), an emotion can be estimated with high accuracy based on the physiological signal.

Particularly, if the emotion-type information table 121 is based on medical evidence, an error (degree of deviation) in a correlation between a physiological signal and an emotion can be reduced.

Moreover, the emotion-type information table 121 can be updated at any time with information extracted from medical evidence. As described, as the emotion-type information table 121 is updated, training of the model is progressed, and the estimation accuracy is further improved.

### Second Embodiment

One example of the result display screen of the emotion estimation has been explained using FIG. 8, but a display form of the result display screen is preferable to be changed to an appropriate form depending on its utilization form. Accordingly, other display forms of the result display screen will be explained next.

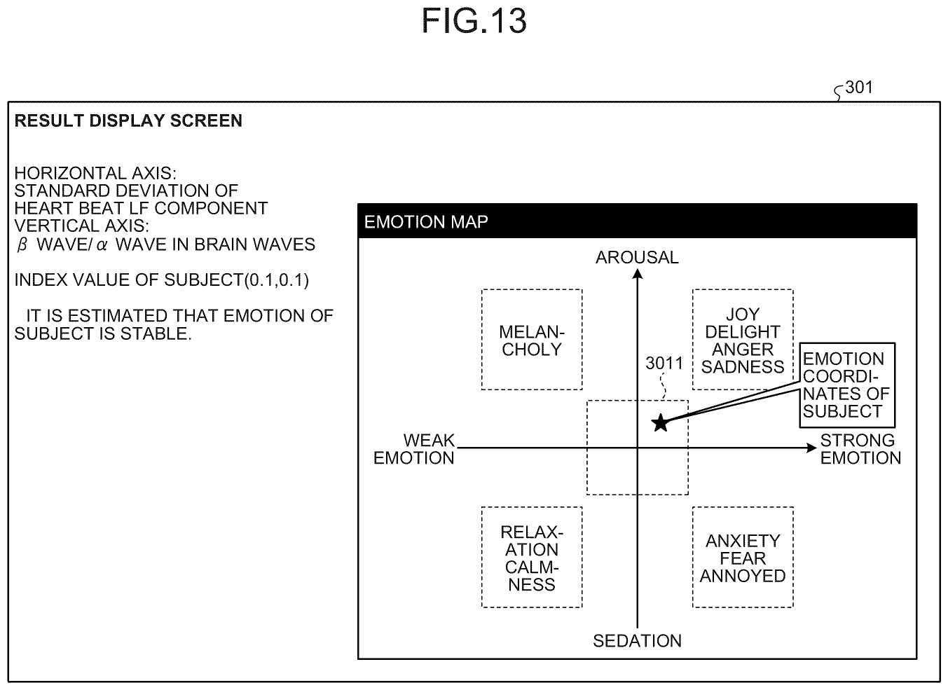

FIG. 13 is a diagram illustrating an example of a result display screen according to a second embodiment. Similarly to FIG. 8, in FIG. 13, an index value of an index representing the arousal is assigned to the vertical axis, and an index value of an index representing the intensity of emotion is assigned to the horizontal axis.

The controller 13 (the providing unit 134) indicates whether an emotion of a subject is in a stable (calm (usual) state) state on the result display screen 301. Specifically, the controller 13 (the providing unit 134) displays a rectangular frame shape 3011 surrounded by a broken line indicating a stable region of emotion, and indicates whether the emotion of the subject is stable based on whether coordinates of an estimated emotion of the subject is present within the frame shape 3011 (inside the frame shape 3011 is the stable-state determination region).

The stable region of emotion is, for example, a region limited by values obtained by accumulating a predetermined coefficients of a maximum value and a minimum value of a vertical axis index value and a horizontal axis index value (defining each of the maximum value and the minimum value for the vertical axis and the horizontal axis), and the predetermined coefficients are set to appropriate values based on experiments or the like.

For example, when the vertical axis index value and the horizontal axis index value are normalized such that the maximum value is 1 and the minimum value is −1, the coefficient described above is set to an appropriate numerical value smaller than 1, such as 0.2. In this case, coordinates of the four vertices of the frame shape 3011 are (0.2, 0.2), (−0.2, 0.2), (0.2, −0.2), (−0.2, −0.2).

For example, if the respective index values are all within a range of −0.2 to 0.2, the controller 13 (the providing unit 134) displays an estimated emotion mark (star in the example in FIG. 10) at an emotion coordinates indicating the estimated emotion in the frame shape 3011 in the emotion stable region. Furthermore, the controller 13 (the providing unit 134) displays a message “EMOTION OF SUBJECT SEEMS STABLE”. As described, according to the second embodiment, it is possible to explicitly indicate whether the emotion of the subject is in a stable state.

In the example described above, it is configured to suggest that the emotion of the subject is in a stable state, but by appropriately setting the position and the size of the frame shape 3011, it becomes possible to suggest whether it is in a specific emotional state. The appropriate position and the size of the frame shape 3011 can be defined by a method in which a range of an emotion coordinates corresponding to a state desired to be confirmed is set based on experiments and the like. For example, when a worker at a factory is regarded as the subject, a range of an emotional state that can be considered that the subject is being focused on a work is set as the frame shape 3011. Moreover, for example, when a driver of a vehicle is the subject, a range of an emotional state that can be considered that the subject is driving calmly is set as the frame shape 3011.

Furthermore, the controller 13 (the providing unit 134) may change the scale of the axis to an appropriate format. For example, the controller 13 (the providing unit 134) can provide a display with high visibility according to a purpose of use, or characteristics of an estimated emotion type or an index type, such as suggesting a specific range of emotion coordinate in detail, by using a nonlinear scale in which the scale becomes smaller as the index value decreases.

### Third Embodiment

There is a case in which it is more appropriate to change a criteria (threshold) for emotion estimation according to a purpose of use of an estimated emotion. For example, when estimation emotion information is used for evaluation of a horror movie, it is conceivable that the evaluation of the movie using the estimated emotion can be performed appropriately by a method in which the threshold for a fear emotion is adjusted based on a targeted level of fear, in which the threshold for the fear emotion is adjusted according to an age of a viewer of the horror movie, or the like.

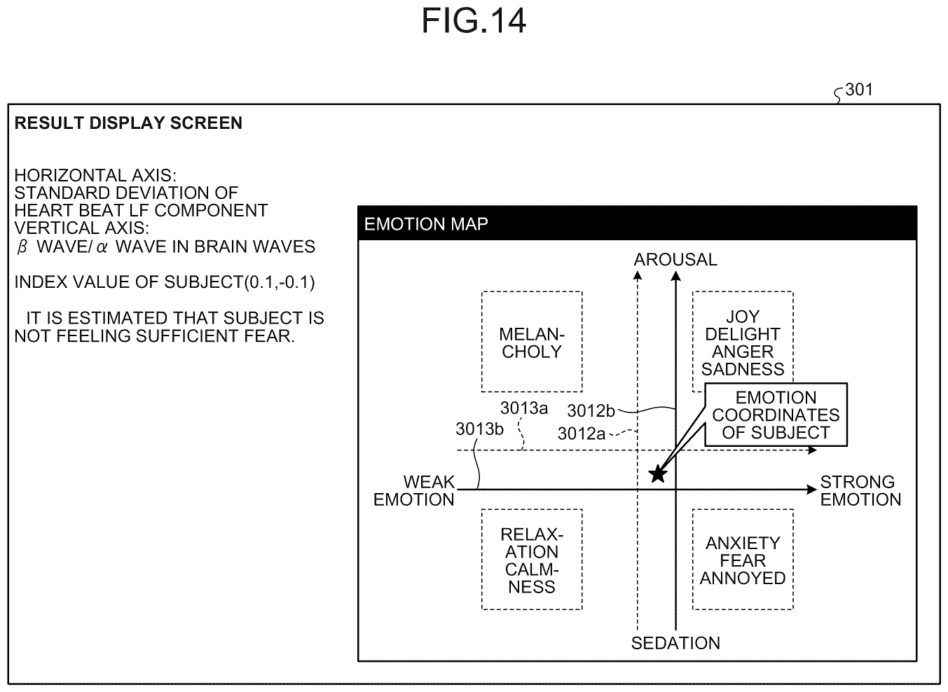

Therefore, in a third embodiment, as illustrated in FIG. 14, by moving a position (origin) of an emotion axis (both of the vertical axis and the horizontal axis, or either one), ranges of respective quadrants for emotion determination are changed.

In normal estimation determination, an emotion map in which a vertical axis 3012a and a horizontal axis 3013a intersect each other at a position at which respective index values are 0, that is, at the origin is used. The vertical axis 3012a and the horizontal axis 3013a are for explanation, and it is not displayed on the result display screen 301 in an actual situation in the third embodiment.

However, for horror movies as described above, in the case of evaluating a movie aiming for a higher level of fear, more appropriate evaluation is possible if a threshold corresponding to a stronger fear than usual is used. That is, depending on a purpose of use, there is a case in which more appropriate emotion estimation can be performed if the positions of the vertical axis and the horizontal axis are moved in the emotion map. Therefore, in the third embodiment, according to a purpose of use of estimated emotion, specifically, based on a type of device that uses the estimated emotion, on an input of the purpose of use by the user, or on an emotion-axis-position adjusting operation by the user, the position of the emotion axis (determination threshold for emotion determination) is changed.

Specifically, the controller 13 (the providing unit 134) moves, when the vertical axis index value and the horizontal axis index value are normalized such that the maximum value is 1 and the minimum value is −1, the vertical axis position and the horizontal axis position according to a purpose of use, for example, by 0.2 toward the negative side, to display on the emotion map of the result display screen 301. That is, the horizontal axis is moved to a position at which the arousal is −0.2 (a horizontal line passing through coordinates (0, −0.2)), and the vertical axis is moved to a position at which the intensity of emotion is +0.2 (a vertical line passing through coordinates (0.2, 0) (intersection coordinates are (0.2, −0.2).

The controller 13 (the providing unit 134) displays a mark (for example, star) at coordinate positions of the respective index values based on physiological signals of the subject. The emotion coordinates of the subject is positioned in the fourth quadrant when the vertical axis 3012a and the horizontal axis 3013a are the reference (before moving axes). Therefore, in this case, it is estimated that the subject is feeling fear.

On the other hand, the emotion coordinates of the subject is positioned in the second quadrant when a vertical axis 3012b and a horizontal axis 3013b are the reference (after moving axes). In this case, it is estimated that the subject is not feeling fear at an intended intensity. In this case, for example, the controller 13 (the providing unit 134) displays a message “IT IS ESTIMATED THAT SUBJECT IS NOT FEEING SUFFICIENT FEAR” on the result display screen 301.

As described, by moving the positions of the vertical axis and the horizontal axis according to a purpose of use of an estimated emotion or the like, a region of each quadrant can be adjusted. That is, an emotion estimation result can be adjusted according to the purpose of use of the estimated emotion. For example, in evaluation of contents, such as a horror movie, evaluation of an emotion (fear) according to an aimed level of fear of creation for the viewer to experience becomes possible.

### About Emotion Type Information

The emotion-type information table 121 illustrated in FIG. 3 may be updated as necessary. For example, in the emotion-type information table 121 illustrated in FIG. 3, a keyword “BOREDOM” is not included in the “POSITIVE-SIDE EMOTION TYPE” and “NEGATIVE-SIDE EMOTION TYPE” in the record of the index ID data “VS01”.

In this case, for a relationship between the emotion “BOREDOM” and brain waves, if medical evidence is newly found, or proofs are provided by experiments or the like, the keyword “BOREDOM” may be added to the relevant side out of “POSITIVE-SIDE EMOTION TYPE” and “NEGATIVE-SIDE EMOTION TYPE” in the record of the index ID data “VS01” based on the medical evidence or contents of the experiments and the like.

Moreover, based on the medical evidence or the like, if a new index suitable for emotion estimation is found, a new data record may be generated for the index and respective data, such as a sensor type corresponding to the use, description, and an emotion axis, may be stored.

More effects and modifications can be derived easily by those skilled in the art. Therefore, a broader aspect of the present invention is not limited to the specific details and representative embodiments denoted an described as above. Therefore, without departing from the spirit or scope of the general concept of the invention defined by the accompanying claims and their equivalents, various modifications are possible.

### REFERENCE SIGNS LIST

N NETWORK
U01 ANALYST
U02 SUBJECT
1 ESTIMATION SYSTEM
10 SERVER
11 COMMUNICATION UNIT
12 STORAGE UNIT
13 CONTROLLER
20 TERMINAL DEVICE
31, 32 SENSOR
121 EMOTION-TYPE INFORMATION TABLE
131 EXTRACTING UNIT
132 GENERATING UNIT
133 IDENTIFYING UNIT
134 PROVIDING UNIT
210, 215, 220, 225, 230, 235, 240, 245 REGION
301 RESULT DISPLAY SCREEN
302 ANALYSIS SUPPORT SCREEN

1. An emotion estimation device to estimate an emotion, comprising:
a controller; and
an emotion estimation model, wherein
in the emotion estimation model, four combination states in which two brain wave states obtained by stratifying a first index based on a ratio of β wave to α wave in a brain wave by a first determination threshold and two heart beat states obtained by stratifying a second index based on a heart beat low-frequency component by a second determination threshold are combined are formed, and an emotion type is set to each of the four combination states, and
the controller
determines the brain wave state by stratifying the first index calculated based on a brain wave acquired from a subject by the first determination threshold,
determines the heart beat state by stratifying the second index calculated based on a heart rate acquired from the subject by the second determination threshold, and
determines the combination state corresponding to the determined brain wave state and heart beat state in the emotion estimation model, and determines an emotion type corresponding to the determined combination state as an estimated emotion.

2. The emotion estimation device according to claim 1, wherein
in the emotion estimation model,
to the combination state in which the first index is larger than the first determination threshold and the second index is larger than the second determination threshold, an emotion type of “joy”, “delight”, “anger”, or “sadness” is set,
to the combination state in which the first index is larger than the first determination threshold and the second index is smaller than the second determination threshold, an emotion type of “melancholy” is set,
to the combination state in which the first index is smaller than the first determination threshold and the second index is smaller than the second determination threshold, an emotion type of “relaxation” or “calmness” is set, and
to the combination state in which the first index is smaller than the first determination threshold and the second index is larger than the second determination threshold, an emotion type of “anxiety”, “fear”, or “annoyed” is set.

3. The emotion estimation device according to claim 1, wherein
the controller adjusts any one of the first determination threshold and the second determination threshold based in an adjustment operation input.

4. The emotion estimation device according to claim 1, wherein
the controller
estimates a purpose of use of an estimated emotion, and
adjusts any one of the first determination threshold and the second determination threshold based on the estimated purpose of use.

5. The emotion estimation device according to claim 1, wherein
the controller
inputs a selection operation to select an index type to be used as either one of the first index and the second index, and
notifies a type of biosensor corresponding to the index type selected by the selection operation.

6. The emotion estimation device according to claim 1, wherein
on a display, the controller
displays an emotion map in which the first index is the vertical axis and the second index is the horizontal axis,
displays a mark image at a coordinate position constituted of the first index and the second index calculated based on physiological signals, in the emotion map, and
displays text information relating to an estimated emotion.

7. An emotion estimation device to estimate an emotion, comprising:
a controller; and
an emotion estimation model, wherein
in the emotion estimation model, four combination states in which two heart beat states obtained by stratifying a first index based on a heart beat interval by a first determination threshold and two heart beat low-frequency states obtained by stratifying a second index based on a heart beat low-frequency component by a second determination threshold are combined are formed, and an emotion type is set to each of the four combination states, and
the controller
determines the heartbeat interval state by stratifying the first index calculated based on a brain wave acquired from a subject by the first determination threshold,
determines the heart beat low-frequency state by stratifying the second index calculated based on a heart rate acquired from the subject by the second determination threshold, and
determines the combination state corresponding to the determined heart beat interval state and heart beat low-frequency state in the emotion estimation model, and determines an emotion type corresponding to the determined combination state as an estimated emotion.

8. The emotion estimation device according to claim 7, wherein
in the emotion estimation model,
to the combination state in which the first index is larger than the first determination threshold and the second index is larger than the second determination threshold, an emotion type of “joy”, “delight”, “anger”, or “anxiety” is set,
to the combination state in which the first index is smaller than the first determination threshold and the second index is smaller than the second determination threshold, an emotion type of “melancholy”, “boredom”, “relaxation”, or “calmness” is set, and
to the combination state in which the first index is smaller than the first determination threshold and the second index is larger than the second determination threshold, an emotion type of “annoyed” or “sadness” is set.

9. A method of generating an emotion estimation model to estimate an emotion, wherein
in the emotion estimation model,
an empty model in which an emotion type to be output as an estimation result to each combination state out of four combination states can be set is formed, the four combination states in which two brain wave states obtained by stratifying a first index based on a ratio of β wave to α wave in a brain wave by a first determination threshold and two heart beat states obtained by stratifying a second index based on a heart beat low-frequency component by a second determination threshold are combined,
a first emotion candidate that is an emotion type experienced in the respective two heart brain wave states is extracted from external information,
a second emotion candidate that is an emotion type experienced in the respective two heart beat states is extracted from external information,
a duplicated emotion type that is duplicated in the first emotion candidate and the second emotion candidate, and the brain wave state and the heart beat state in which the duplicated emotion type is included as the first emotion candidate and the second emotion candidate are extracted, and
the duplicated emotion type is set to a combination state in the empty model corresponding to the respective extracted brain wave state and heart beat state.

10. The method of generating an emotion estimation model according to claim 9, wherein
the first index is determined based on information about a relationship between a ratio of β wave to α wave in a brain wave and an emotion extracted from external information, and
the second index is determined based on information about a relationship between a heart beat low-frequency component and an emotion extracted from external information.
